(19) United States
(12) Patent Application Publication
(10) Pub. No.: US 2016/0058272 A1
(43) Pub. Date: Mar. 3, 2016

(54) CAPSULE ENDOSCOPE, CAPSULE ENDOSCOPE SYSTEM, AND METHOD FOR CONTROLLING POSTURE OF CAPSULE ENDOSCOPE

(71) Applicant: Panasonic Intellectual Property Management Co., Ltd., Osaka (JP)

(72) Inventors: ATSUSHI OMOTE, Osaka (JP); TAKAYUKI ISHIKAWA, Osaka (JP); KENJI TAGASHIRA, Osaka (JP); MASATO ISHINO, Osaka (JP); KENICHI NISHIUCHI, Osaka (JP)

(21) Appl. No.: 14/731,737

(22) Filed: Jun. 5, 2015

(30) Foreign Application Priority Data
Aug. 28, 2014 (JP) 2014-173567

(51) Int. Cl. A61B 1/04; A61B 1/00

(52) U.S. Cl. CPC A61B 1/041; A61B 1/00147; A61B 1/00006; A61B 1/00045

(57) ABSTRACT

A capsule endoscope includes a capsule enclosure having an external wall surface; an image pickup device provided inside the capsule enclosure; a light source provided inside the capsule enclosure; a plurality of electrode structures each including an electrode, a water repellent layer, and a dielectric layer positioned between the electrode and the water repellent layer, the plurality of electrode structures being provided on the external wall surface of the capsule enclosure such that the electrode is positioned on an external wall surface side of the capsule enclosure; a power supply provided inside the capsule enclosure; at least one reference electrode provided on the external wall surface of the capsule enclosure and connected to reference potential of the power supply; and a drive circuit configured to apply a drive voltage to the plurality of electrode structures based on the power supply.

### BACKGROUND

1. Technical Field

The present application relates to a capsule endoscope, a capsule endoscope system, and a method for controlling posture of a capsule endoscope.

2. Description of the Related Art

Capsule endoscopes that have been put into practical use each incorporate a small camera for photographing an organ of a digestive system. Conventional capsule endoscopes move by peristaltic motion of an organ. Patent Literatures 1 and 2 each disclose a capsule endoscope that can move under its own power.

### CITATION LIST

### Patent Literatures

PTL 1: WO2014/014062

PTL 2: Unexamined Japanese Patent Publication No. 2014-36723

### SUMMARY

An object of a capsule endoscope is to examine a digestive organ, and it is desired to pick up an image of a desired portion inside a living body. A capsule endoscope according to a non-limiting embodiment of the present application provides a novel capsule endoscope capable of controlling posture.

A capsule endoscope according to one embodiment of the present disclosure includes: a capsule enclosure having an external wall surface; an image pickup device provided inside the capsule enclosure; a light source provided inside the capsule enclosure; a plurality of electrode structures each including an electrode, a water repellent layer, and a dielectric layer positioned between the electrode and the water repellent layer, the plurality of electrode structures being provided on the external wall surface of the capsule enclosure such that the electrode is positioned on an external wall surface side of the capsule enclosure; a power supply provided inside the capsule enclosure; at least one reference electrode provided on the external wall surface of the capsule enclosure and connected to reference potential of the power supply; and a drive circuit configured to apply a drive voltage to the plurality of electrode structures based on the power supply.

The capsule endoscope disclosed in the present application allows for posture control of the capsule endoscope by using electrowetting.

### BRIEF DESCRIPTION OF DRAWINGS

FIG. 1A is an external appearance schematically illustrating a capsule endoscope according to a present embodiment;

FIG. 1B is a diagram schematically illustrating a configuration of the capsule endoscope according to the present embodiment;

FIG. 1C is a cross-sectional view illustrating an electrode structure;

FIG. 1D is a block diagram illustrating a configuration of an operation unit according to the present embodiment;

FIG. 2A is a diagram illustrating coordinates for description of the capsule endoscope;

FIG. 2B is a three-way diagram illustrating arrangement of the electrode structures of the capsule endoscope according to the present embodiment;

FIG. 3A is a diagram illustrating a circuit configuration and a force applied by EW (electrowetting) in a case where a direct-current (DC) voltage is applied to the electrode structures;

FIG. 3B is a diagram illustrating a circuit configuration and a force applied by EW in a case where an alternating-current (AC) voltage is applied to the electrode structures;

FIG. 3C is a diagram illustrating waveforms of a drive voltage applied to respective terminals and a waveform of a potential difference applied to the electrode structure to drive in a case where an AC voltage is applied to the electrode structures;

FIG. 3D is a diagram illustrating waveforms of another drive voltage applied to respective terminals and a waveform of a potential difference applied to the electrode structure to drive in a case where an AC voltage is applied to the electrode structures;

FIG. 4 is a diagram illustrating a relationship between the voltage applied to the electrode structures and posture control directions;

FIG. 5A is a diagram illustrating a positional relationship between a reference electrode and electrode structure of the capsule endoscope, and an organ and body fluid of a subject;

FIG. 5B is a diagram illustrating a positional relationship between the reference electrode and electrode structure of the capsule endoscope, and the organ and body fluid of the subject;

FIG. 6 is a three-way diagram illustrating another arrangement of the electrode structures of the capsule endoscope;

FIG. 7 is a three-way diagram illustrating another arrangement of the reference electrodes of the capsule endoscope;

FIG. 8A is a diagram illustrating an example of the capsule endoscope including a sampling pipe;

FIG. 8B is a cross-sectional view illustrating structure of the sampling pipe;

FIG. 9 is a schematic view of an example illustrating a cell using electrowetting;

FIG. 10A is a diagram illustrating liquid level variation of the cell of the example in a case where a voltage of 0 V is applied;

FIG. 10B is a diagram illustrating liquid level variation of the cell of the example in a case where a voltage of 150 V is applied; and

FIG. 11 is a diagram illustrating a rigid body rotating around a rotational axis perpendicular to a bar at a center of the bar.

### DETAILED DESCRIPTION OF THE EMBODIMENT

Capsule endoscopes disclosed in Patent Literature 1 and Patent Literature 2 use vibration of a motor or a coil in order to self-travel and to perform posture control. These drive mechanisms consume relatively large electric power. However, when a power supply is mounted inside the capsule endoscope, since the capsule endoscope has a limited size and usable capacity of the power supply cannot be large, the capsule endoscope may fail to self-travel and to perform posture control for a long time.

In addition, it is necessary to provide a mechanical component for generating a driving force, such as a blade or a screw, outside an enclosure of the capsule endoscope.

Accordingly, from a viewpoint of reduction in invasiveness, it is considered that the capsule endoscope has a problem such that the mechanical component becoming an obstacle when the capsule endoscope is swallowed or discharged.

In view of such problems, the present inventors have conceived application of hydrophilic properties and water repellency of an electrode produced by an electrowetting technique to posture control of the capsule endoscope. An outline of the capsule endoscope according to the present disclosure is as follows.

A capsule endoscope according to one embodiment of the present disclosure includes: a capsule enclosure having an external wall surface; an image pickup device provided inside the capsule enclosure; a light source provided inside the capsule enclosure; a plurality of electrode structures each including an electrode, a water repellent layer, and a dielectric layer positioned between the electrode and the water repellent layer, the plurality of electrode structures being provided on the external wall surface of the capsule enclosure such that the electrode is positioned on an external wall surface side of the capsule enclosure; a power supply provided inside the capsule enclosure; at least one reference electrode provided on the external wall surface of the capsule enclosure and connected to reference potential of the power supply; and a drive circuit configured to apply a drive voltage to the plurality of electrode structures based on the power supply.

The drive circuit may change hydrophilic properties on a surface of the water repellent layer of each of the electrode structures by controlling the drive voltage to be applied to the plurality of electrode structures.

The external wall surface of the capsule enclosure may have a longitudinal direction and a shape of a rotating body rotating around a rotational axis parallel to the longitudinal direction. The external wall surface may have first and second regions divided by a plane perpendicular to the rotational axis.

The plurality of electrode structures may include at least two electrode structures arranged in the first and second regions, respectively.

The plurality of electrode structures may include: a first electrode structure arranged in the first region; and second, third, fourth, and fifth electrode structures arranged in a circumferential direction of the external wall surface in the second region.

The external wall surface of the capsule enclosure may have a longitudinal direction and a shape of a rotating body rotating around a rotational axis parallel to the longitudinal direction. The image pickup device may be positioned on a one-end side in the longitudinal direction of the capsule enclosure.

The external wall surface of the capsule enclosure may include eight regions divided by a first plane perpendicular to the rotational axis, and by a second plane and a third plane including the rotational axis and being orthogonal to each other. The plurality of electrode structures may include first, second, third, and fourth electrode structures arranged in four regions on a side of the first plane where the image pickup device is positioned, respectively, in a clockwise order when the external wall surface of the capsule enclosure is viewed along the rotational axis from a side where the image pickup device is positioned. The plurality of electrode structures may also include fifth, sixth, seventh, and eighth electrode structures arranged in four regions on an opposite side of the first plane from the image pickup device, the fifth, sixth, seventh, and eighth electrode structures being adjacent to the first, second, third, and fourth electrode structures, respectively.

The at least one reference electrode may include two reference electrodes, and the two reference electrodes may be arranged at both ends in the longitudinal direction of the external wall surface, respectively.

The at least one reference electrode may have a shape of a belt.

The first, second, and third reference electrodes may be positioned on the first, second, and third planes, respectively.

The drive voltage may be a direct-current voltage.

The drive circuit may include a booster circuit configured to generate the drive voltage higher than a voltage of the power supply based on the power supply. The drive circuit may also include a relay including a first end connected to a terminal to which the booster circuit outputs the drive voltage, and a second end connected to the electrode of each of the electrode structures.

The drive voltage may be an alternating-current voltage.

The drive circuit may include: a DC/AC converter configured to generate the alternating-current voltage based on the power supply, and to apply the alternating-current voltage to each of the electrode structures; and a phase controller configured to control a phase of the alternating-current voltage to be applied to each of the electrode structures.

The capsule endoscope may further include: a sampling pipe provided inside the capsule enclosure, the sampling pipe having an opening on the external wall surface of the capsule enclosure; and a different electrode structure including an electrode, a water repellent layer, and a dielectric layer positioned between the electrode and the water repellent layer, the different electrode structure being provided on an inner wall of the sampling pipe such that the electrode is positioned on the inner wall of the sampling pipe.

The capsule endoscope may further include a controller and a wireless communicator provided inside the capsule enclosure. The wireless communicator may transmit image data obtained by the image pickup device to an external apparatus, and may receive a control signal from the external apparatus. The controller may drive the drive circuit in response to the control signal, and may apply the drive voltage to the plurality of electrode structures selectively.

A capsule endoscope system according to one embodiment of the present disclosure includes the above-described capsule endoscope and an operation unit. The operation unit includes: a different wireless communicator configured to receive the image data transmitted from the capsule endoscope and to transmit the control signal; an image processor configured to apply image processing to the image data received by the different wireless communicator; a display unit configured to display the image data that undergoes the image processing; an input device configured to receive an input from an operator; and a control signal generator configured to generate the control signal in response to the input to the input device.

A method for controlling posture of a capsule endoscope according to one embodiment of the present disclosure includes: providing a plurality of electrode structures on an external wall surface of the capsule endoscope including a capsule enclosure, each of the electrode structures including an electrode, a water repellent layer, and a dielectric layer positioned between the electrode and the water repellent layer, the plurality of electrode structures being provided on the external wall surface of the capsule enclosure, such that the electrode is positioned on an external wall surface side of the capsule enclosure; and changing hydrophilic properties on a surface of the water repellent layer of each of the electrode structures, and changing posture of the capsule enclosure, by applying a drive voltage to the plurality of electrode structures.

An embodiment of a capsule endoscope and a capsule endoscope system will be described below.

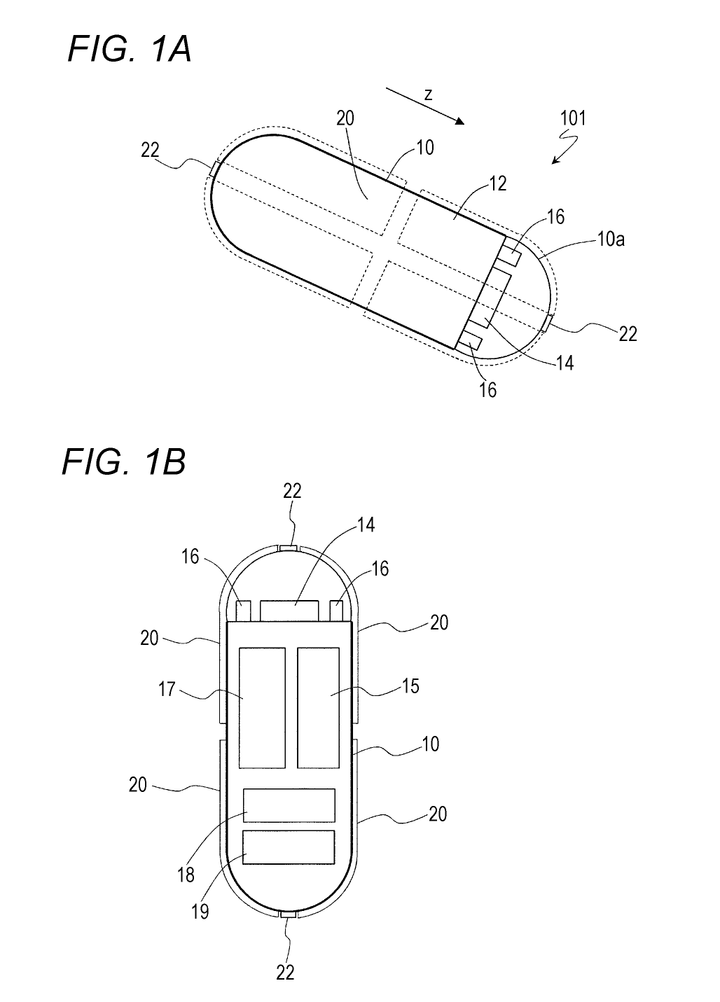

FIG. 1A schematically illustrates an external appearance of capsule endoscope 101 according to the present embodiment. FIG. 1B schematically illustrates a configuration of capsule endoscope 101.

Capsule endoscope 101 includes capsule enclosure 10, image pickup device 14, controller 15, light source 16, wireless communicator 17, power supply 18, drive circuit 19, electrode structures 20, and reference electrode 22.

Capsule enclosure 10 includes internal space, can be swallowed by a subject from a mouth, and has a size suitable to pass through a digestive organ of a human body. For example, an external wall surface of capsule enclosure 10 has a longitudinal direction z and a shape of a rotating body rotating around a rotational axis parallel to the longitudinal direction z. More specifically, the external wall surface of capsule enclosure 10 has a circular cross-section perpendicular to the rotational axis, and the cross-section has a diameter ranging from about 5 mm to about 15 mm. The longitudinal direction z has a length ranging from about 10 mm to about 35 mm.

Capsule enclosure 10 is formed of a material that is not invaded by acids or enzymes in a living body, such as resin or metal. In addition, in order to pick up an image, portion 10a of capsule enclosure 10 is formed of various resins transparent to visible light. According to the present embodiment, portion 10a is positioned at one end of the longitudinal direction z of capsule enclosure 10. However, portion 10a may be provided depending on a position at which image pickup device 14 is provided. For example, portion 10a may be provided in a vicinity of a center in the longitudinal direction z of capsule enclosure 10.

Image pickup device 14 includes an optical system such as a lens, an image sensor, and an image processing circuit, and is provided in a vicinity of an end of the longitudinal direction z inside capsule enclosure 10. Image pickup device 14 photographs still images at predetermined time intervals, or shoots moving images. Image pickup device 14 may photograph still images or shoot moving images at a timing based on an instruction from controller 15.

Controller 15 controls operation of respective units of capsule endoscope 101.

Light source 16 is provided inside capsule enclosure 10 and adjacent to image pickup device 14. Light source 16 emits illumination light. The plurality of light sources 16 may be provided in such a way that illumination light may be uniformly distributed across a region that image pickup device 14 can photograph. When images are photographed with visible light, for example, white illumination light is used. When images are photographed with infrared rays, ultraviolet rays, or the like, light source 16 that emits corresponding rays is used.

Wireless communicator 17 transmits image data obtained by image pickup device 14 to an external operation unit in real time, as will be described in detail below. In addition, wireless communicator 17 receives posture control data for capsule endoscope 101 from the operation unit.

Power supply 18 supplies electric power for operating respective units of capsule endoscope 101. Power supply 18 is, for example, a lithium-ion battery, and is a DC power supply of several volts.

Drive circuit 19 generates a drive voltage to be applied to electrode structures 20, and applies the drive voltage to electrode structures 20 in accordance with control by controller 15. This will change an affinity (hydrophilic properties/water repellency) of surfaces of electrode structures 20 for water.

Electrode structures 20 are provided on the external wall surface of capsule enclosure 10. FIG. 1C schematically illustrates detailed structure of electrode structures 20. As illustrated in FIG. 1C, each of electrode structures 20 includes electrode 25, water repellent layer 27, and dielectric layer 26 positioned between electrode 25 and water repellent layer 27. Electrode structures 20 are provided on the external wall surface such that electrode 25 may be positioned on an external wall surface side of capsule enclosure 10. Electrode 25 has electric conductivity. Electrode 25 is formed of, for example, various metallic materials, such as Al, Pt, Al, Ag, and Cu. As described above, when electrode structures 20 are provided to cover portion 10a, electrode structures 20 may be formed of a transparent conductive material, such as ITO or ZnO, so as to avoid obstructing a photographing range of image pickup device 14. Electrode 25 preferably has good adhesive properties with capsule enclosure 10 and dielectric layer 26. Therefore, electrode 25 may have laminated structure of a layer made of Cr or Ti and a layer made of the above-described material as necessary.

Electrode 25 can be formed by using a forming method such as an evaporation method or a sputtering method. When electrode 25 is thick, a height difference from the external wall surface of capsule enclosure 10 increases, resulting in variations in thicknesses of dielectric layer 26 and water repellent layer 27. In consideration of securing sufficient conductivity, the thickness of electrode 25 is preferably between not less than 0.01 μm and not more than 1 μm.

Dielectric layer 26 can be formed of various insulating materials having little influence on a human body and body fluid. Examples of insulating materials that can be used include various macromolecular compounds, various oxides of inorganic compounds, composite oxides, and nitrides. If a dielectric breakdown occurs in dielectric layer 26 when the drive voltage is applied, current leakage causes an electric current to flow through a body of a subject, or inhibits posture control of capsule endoscope 101. Accordingly, a dielectric substance to be used as a material of dielectric layer 26 needs to have a dielectric breakdown voltage high enough to endure the applied drive voltage. Dielectric layer 26 that is thicker than necessary for a purpose of securing the dielectric breakdown voltage will require high drive voltage for posture control. For this reason, the thickness of dielectric layer 26 is preferably 1 μm or less.

When a macromolecular compound is used as a material of dielectric layer 26, dielectric layer 26 can be formed by methods such as a dipping method, a spray coating method, and a spin coating method. When an inorganic compound is used as a material of dielectric layer 26, dielectric layer 26 can be formed by a method such as a sputtering method, a spray coating method, or a spin coating method. The plurality of electrode structures 20 are arranged on the external wall surface of capsule enclosure 10. Accordingly, if the thickness of dielectric layer 26 differs greatly between the plurality of electrode structures 20, degree of hydrophilic properties on surfaces of electrode structures 20 may differ even if a common drive voltage is applied. Therefore, variations in the thickness of dielectric layer 26 are preferably within approximately ±10% between the plurality of electrode structures 20, and within one electrode structure 20.

Water repellent layer 27 can be formed by using various organic compounds having little influence on a human body and body fluid. For example, compounds having fluoroalkyl chains, such as polytetrafluoroethylene (PTFE) or AF1600 (produced by Du Pont), typically have high water repellency, and can be particularly preferably used. Among various organic compounds, a compound having a silane coupling group produces a coupling reaction with dielectric layer 26 and provides high adhesive properties, and thus can be particularly preferably used. Organic compounds that can form a silane coupling and that have high water repellency include organic compounds having fluoro-alkyl chains. Examples of such organic compounds are trifluoropropyltrimethoxysilane, perfluorooctyltrimethoxysilane, perfluorodecyltrimethoxysilane, perfluorooctyltrichlorosilane, and perfluorodecyltrichlorosilane. As a macromolecular material having a silane coupling group, for example, products such as CYTOP (produced by Asahi Glass), Optool (produced by Daikin Industries) are commercially available. These macromolecular materials allow for easy control of film thickness, and thus can be used particularly preferably.

Water repellent layer 27 may have a high dielectric breakdown voltage, in a similar manner to dielectric layer 26. Meanwhile, water repellent layer 27 that is thicker than necessary for securing the dielectric breakdown voltage will lead to higher drive voltage. Accordingly, the thickness of water repellent layer 27 is preferably 2 μm or less. Water repellent layer 27 can be formed by using a method such as a dipping method, a spray coating method, or a spin coating method. When water repellent layer 27 that requires chemical reactions is used, such as a silane coupling agent and heat curing, heat treatment may be applied as necessary after consideration of heat resistance of capsule enclosure 10, electrode 25, and dielectric layer 26. Variations in the thickness of water repellent layer 27 is preferably within approximately ±10% between the plurality of electrode structures 20 and within one electrode structure 20, in a similar manner to dielectric layer 26.

At least one reference electrode 22 is provided on the external wall surface of capsule enclosure 10. Preferably, reference electrode 22 is adjacent to all of the plurality of electrode structures 20. Reference electrode 22 is formed of various metallic materials or transparent conductive materials, similar to the material of electrode 25. The thickness of reference electrode 22 is also preferably similar to the thickness of electrode 25. Reference electrode 22 is connected to reference potential (0 V) of power supply 18 of capsule endoscope 101. During examination, contact between reference electrode 22 of capsule endoscope 101 and body fluid inside a body of a subject allows the reference potential of capsule endoscope 101 to be identical to potential inside the body. This causes the drive circuit of capsule endoscope 101 to go into a floating state, and inhibits possible application of a voltage higher than the drive voltage to inside the body. Note that reference electrode 22 may not be provided depending on a drive method for posture control described later and on structure of electrode structures 20.

FIG. 1D illustrates a configuration of operation unit 102. Operation unit 102 and capsule endoscope 101 constitute the capsule endoscope system. Operation unit 102 includes wireless communicator 71, image processor 72, display unit 73, control signal generator 74, input device 75, and memory 76.

Wireless communicator 71 and wireless communicator 17 of capsule endoscope 101 communicate with each other. Specifically, wireless communicator 71 receives image data transmitted from wireless communicator 17 of capsule endoscope 101. Operation unit 102 includes different wireless communicator 77 that a subject can carry when wireless communicator 17 of capsule endoscope 101 transmits a small output. Wireless communicator 77 may receive a signal from wireless communicator 17 of capsule endoscope 101, boost the signal, and transmit an output.

The image data transmitted from wireless communicator 17 of capsule endoscope 101 is received in real time by wireless communicator 71. The received image data undergoes adjustment of brightness, contrast, distortion of the image, and the like by image processor 72 so as to become suitable for display, and is displayed on display unit 73. The image data may be stored in memory 76.

An operator observes the image of inside the body of the subject displayed on display unit 73, and controls posture of capsule endoscope 101 as necessary. Specifically, the operator inputs a direction in which the posture of capsule endoscope 101 is to be changed by using input device 75 such as a mouse, a key board, a trackball, or a joy stick. In response to the input from input device 75, control signal generator 74 generates a control signal for changing the posture of capsule endoscope 101, and outputs the control signal to wireless communicator 71. In response to the input from input device 75, image processor 72 may generate an image indicating the posture-changing direction that is input by the operator, superimpose the image on an image of inside the body of the subject, and display the superimposed image on display unit 73.

Wireless communicator 71 transmits the posture-changing control signal to capsule endoscope 101. In response to the posture-changing control signal received by wireless communicator 17, controller 15 of capsule endoscope 101 causes drive circuit 19 to generate the drive voltage. This causes the drive voltage to be applied to electrode structures 20 such that the posture may be changed as the operator intends.

Next, electrode structures 20 in capsule endoscope 101 according to the present embodiment will be described. When at least two electrode structures 20 are provided on the external wall surface of capsule endoscope 101, the two electrode structures 20 can have different affinity for water, which makes it possible to change the posture of capsule endoscope 101. In order to change the posture of capsule endoscope 101 more accurately, four electrode structures are preferably provided in each of two regions obtained by dividing the external wall surface of capsule enclosure 10 in the longitudinal direction. This allows an end of the longitudinal direction to pivot vertically and horizontally when capsule endoscope 101 is viewed from the other end, so that it becomes possible to pick up an image in a desired direction inside the body.

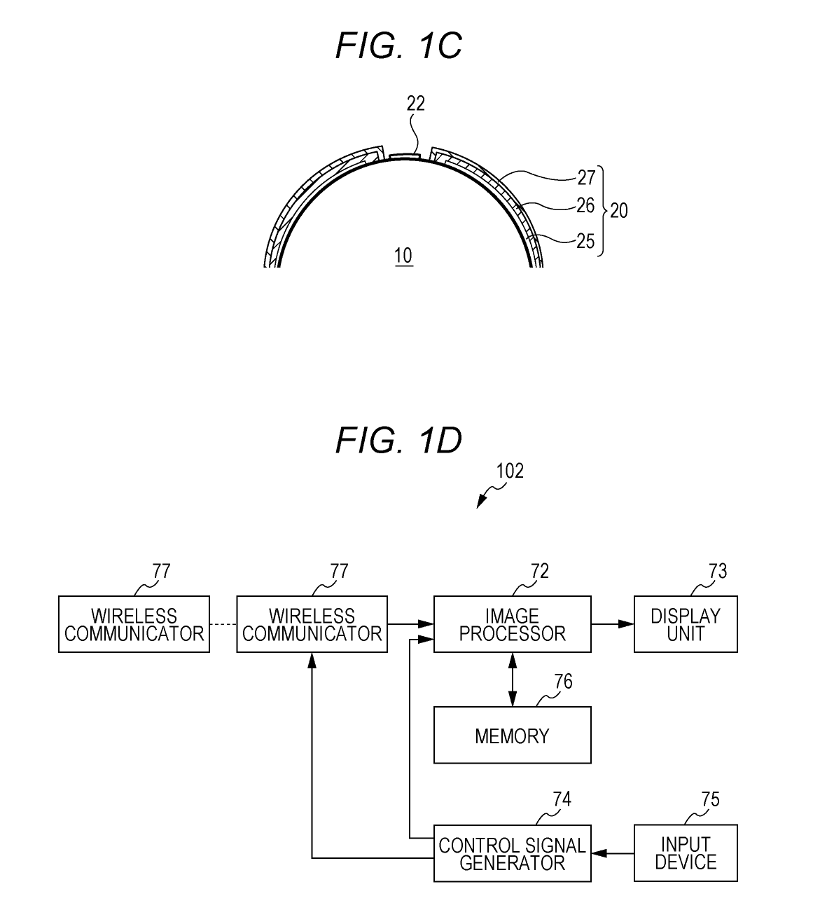

For this purpose, capsule endoscope 101 according to the present embodiment includes eight electrode structures 20. As illustrated in FIG. 2A, the longitudinal direction of capsule enclosure 10 of capsule endoscope 101 is defined as a z-axis direction, whereas an x direction and a y direction are defined in a plane perpendicular to the z-axis direction. The z axis is the rotational axis of capsule enclosure 10. The image pickup device is positioned on a one end El side in the longitudinal direction of capsule enclosure 10.

An upper diagram of FIG. 2B is a diagram of capsule enclosure 10 of capsule endoscope 101 viewed along the z direction. Two lower diagrams of FIG. 2B are diagrams of capsule enclosure 10 of capsule endoscope 101 viewed along the x direction and the y direction.

The external wall surface of capsule endoscope 101 according to the present embodiment is divided into eight regions, and eight electrode structures 20 are positioned in the divided eight regions, respectively. Specifically, the external wall surface of capsule enclosure 10 has eight regions divided by first plane F1 perpendicular to the rotational axis (z axis), and by second plane F2 and third plane F3 that include the rotational axis and are orthogonal to each other.

Among the eight regions, first electrode structure 20NA, second electrode structure 20NB, third electrode structure 20NC, and fourth electrode structure 20ND are positioned in four regions on a side of first plane F1 where the image pickup device is positioned (E1), respectively, in a clockwise order when the external wall surface of capsule enclosure 10 is viewed along the z axis from the side (El side) where the image pickup device is positioned.

In addition, fifth electrode structure 20SA, sixth electrode structure 20SB, seventh electrode structure 20SC, and eighth electrode structure 20SD are positioned in four regions on an opposite side (E2) of first plane F1 from the image pickup device. Fifth electrode structure 20SA, sixth electrode structure 20SB, seventh electrode structure 20SC, and eighth electrode structure 20SD are adjacent to first electrode structure 20NA, second electrode structure 20NB, third electrode structure 20NC, and fourth electrode structure 20ND, respectively. The eight electrode structures are spaced from each other, and are not electrically connected to each other. Accordingly, it is possible to apply the drive voltage to these electrode structures independently. In the following description, the plurality of electrode structures may generically be denoted as electrode structure 20.

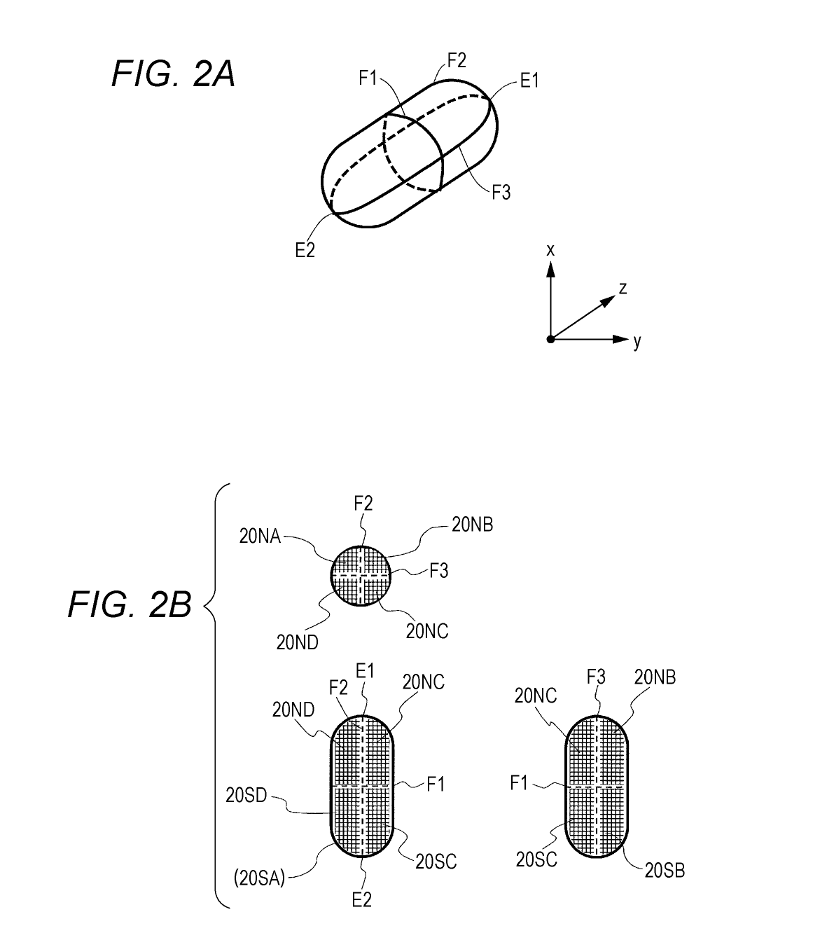

Subsequently, drive circuit 19 and driving of capsule endoscope 101 will be described. FIG. 3A illustrates part of drive circuit 19 when capsule endoscope 101 is driven with a DC voltage. Drive circuit 19 generates a DC drive voltage. For this purpose, drive circuit 19 includes booster circuit 19a, resistor 19b, diode 19c, and relay 19d. Booster circuit 19a increases and outputs a voltage that is output from power supply 18. Booster circuit 19a outputs, for example, a DC voltage ranging from several tens of volts to hundred and several tens of volts.

A negative side of booster circuit 19a is used as a reference voltage, and is connected to reference electrode 22. A positive side of booster circuit 19a is connected to respective electrode structures 20 via relay 19d. An instruction from controller 15 switches relay 19d and selects electrode structure 20 to which the drive voltage is to be applied. For example, FIG. 3A illustrates a state where the drive voltage is applied to electrode structure 20NB, and where the drive voltage is not applied to electrode structure 20NA.

In the state where the drive voltage is applied, reference electrode 22 is electrically connected to electrode structure 20NB by body fluid 30, and an electrostatic charge is accumulated in dielectric layer 26 and water repellent layer 27 of electrode structure 20NB. This makes the surface of electrode structure 20NB hydrophilic. The surface of electrode structure 20NA exhibits water repellency because the drive voltage is not applied. For this reason, as illustrated in FIG. 3A, a force is applied to capsule endoscope 101 in a direction from electrode structure 20NA to electrode structure 20NB, that is, rightward in FIG. 3A.

In order to control hydrophilic properties by electrowetting, the electric charge accumulated in dielectric layer 26 of electrode structure 20 is used, as described above. Electrode structure 20 to which the drive voltage is not applied preferably discharges the accumulated electric charge immediately to be set at potential identical to the reference voltage. For this purpose, diode 19c and resistor 19b connected in parallel with relay 19d and booster circuit 19a are provided for each electrode structure 20. Resistor 19b is connected to the reference potential. When relay 19d is turned off, this circuit configuration causes the electric charge accumulated in dielectric layer 26 and water repellent layer 27 to be discharged through diode 19c and resistor 19b. When relay 19d is turned off, this circuit configuration inhibits rapid electric discharge, and allows for cancellation of the electric charge from the reference electrode side. When relay 19d is turned on, the drive voltage from booster circuit 19a can be applied to electrode structure 20 through diode 19c.

It is also considered that relay 19d connected to electrode structure 20 switches a positive side and a negative side (via resistor 19b) of booster circuit 19a. However, when a large electrostatic charge is accumulated in electrode structure 20, sparks may be produced when relay 19d is switched.

The drive voltage generated by booster circuit 19a depends on the thicknesses of dielectric layer 26 and water repellent layer 27. A discharge current depends on the drive voltage, relative dielectric constants of dielectric layer 26 and water repellent layer 27, and an area of electrode 25. While larger resistor 19b can inhibit a peak of the discharge current, electric discharge needs longer time, and thus posture control also needs longer time. Conversely, smaller resistor 19b reduces the discharge time and accelerates posture control, but the peak of the discharge current will increase. A value of resistor 19b can be selected between not less than 1 kΩ and not more than 10 MΩ, in consideration of the drive voltage, the thicknesses and relative dielectric constants of dielectric layer 26 and water repellent layer 27, the area of electrode 25, and time needed for posture control.

FIG. 3B illustrates part of the drive circuit when capsule endoscope 101 is driven with an AC voltage. Drive circuit 19 generates an AC drive voltage. For this purpose, drive circuit 19 includes DC/AC converter 19e and phase controller 19f. DC/AC converter 19e generates an AC voltage from power supply 18. The drive voltage that is output from DC/AC converter 19e is applied to reference electrode 22 and electrode structures 20. Phase controller 19f controls a phase of the applied drive voltage.

Reference voltage E0, which is an AC voltage, is applied to reference electrode 22, for example. When AC drive voltage E1 having a phase identical to a phase of reference voltage EU is applied to electrode structure 20, potential difference becomes zero and substantially no voltage is applied. Meanwhile, when an AC drive voltage E2 having a phase different from the phase of reference voltage E0 is applied to electrode structure 20, potential difference corresponding to E2-E1 will be applied. For example, when a phase difference between E2 and E1 is 180° , as illustrated in FIG. 3C, the applied voltage has peak amplitude twice as large as peak amplitude of E0. Thus, the effective drive voltage applied to electrode structure 20 can be switched only by controlling the phase of the drive voltage that is output from DC/AC converter 19e. In addition, the posture of capsule endoscope 101 can be controlled by using a voltage of one half of the drive voltage required to change the affinity of the surface of electrode structure 20 for water by electrowetting.

When the above-mentioned ITO or ZnO is used in reference electrode 22, application of a negative voltage of an AC voltage to reference electrode 22 may cause reduction of ITO or ZnO due to an electrochemical reaction, and may change electric conductivity. In this case, as illustrated in FIG. 3D, positive AC voltages may be used for reference voltage E0, drive voltage E1, and drive voltage E2. This can inhibit reduction of ITO or ZnO.

Thus, in a case where electrode structure 20 is driven with a DC voltage, components such as a relay, a diode, or a resistor are needed in drive circuit 19, which may complicate an internal circuit of capsule endoscope 101. In contrast, in a case where electrode structure 20 is driven with an AC voltage, only control of the phase of the drive voltage is needed, and thus the internal circuit can be simplified. In addition, the drive voltage to be generated can be reduced to one half of a voltage value required for posture control. However, in the case of AC drive, the AC voltage may be applied to inside the body.

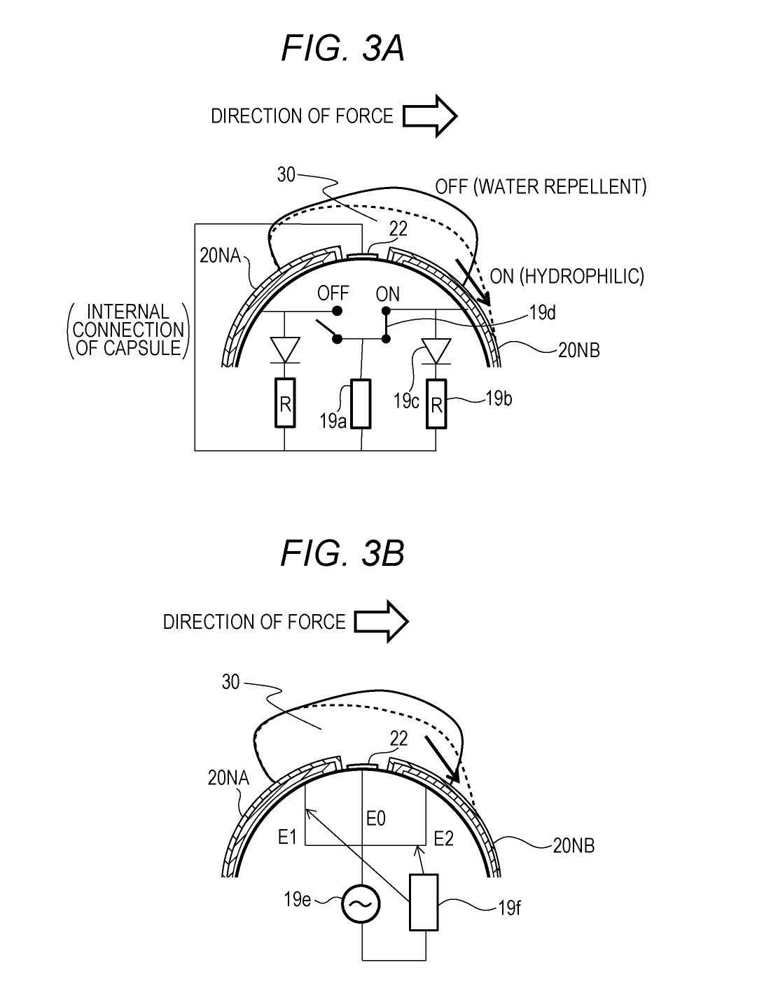

Next, an example of posture control of capsule endoscope 101 will be described with reference to FIG. 4. An upper part of FIG. 4 illustrates directions in which the posture of capsule endoscope 101 is moved. A lower part of FIG. 4 illustrates, as illustrated in FIG. 2B, positions of the electrodes viewed from z, x, and y directions by using the coordinate system illustrated in FIG. 2A. In these diagrams, deep hatching represents an (ON) electrode structure to which the drive voltage is applied, whereas light hatching represents an (OFF) electrode structure to which the drive voltage is not applied.

A dashed-line arrow represents a moving direction of an upper part of capsule endoscope 101, whereas a solid-line arrow represents a moving direction of a lower part of capsule endoscope 101. Since the surface of the electrode structure to which the drive voltage is applied exhibits hydrophilic properties, a force is applied to a side of the electrode structure to which the drive voltage is applied. FIG. 4 illustrates an example of a method for applying the drive voltage for controlling the posture in four directions including rightward, backward, leftward, and frontward directions in accordance with this EW principle. As illustrated in FIG. 4, with respect to posture control of the upper part (traveling direction), the posture of the lower part is controlled such that the drive voltage is applied to the lower part oppositely to the upper part. In other words, the drive voltage is applied to two adjacent electrode structures selected from among first, second, third, and fourth electrode structures. In addition, the drive voltage is applied to two adjacent electrode structures selected from among fifth, sixth, seventh, and eighth electrode structures. The two selected adjacent electrode structures in the upper part and the two selected adjacent electrode structures in the lower part have point symmetry relative to a center of capsule enclosure 10. This causes the forces to be applied to the lower part and the upper part in opposite directions with respect to the center of capsule endoscope 101, and thus posture control becomes easy. Table 1 below shows a state of application of the drive voltage to each electrode structure and corresponding posture control. In Table 1, for ease of viewing, OFF is not shown in (OFF) electrode structure to which the drive voltage is not applied.

TABLE 1

| | POSTURE CONTROL | | | |
|---|---|---|---|---|
| ELECTRODE | RIGHT- | BACK- | LEFT- | FRONT- |
| STRUCTURE | WARD | WARD | WARD | WARD |
| 20NA | | ON | ON | |
| 20NB | ON | ON | | |
| 20NC | ON | | | ON |
| 20ND | | | ON | ON |
| 20SA | ON | | | ON |
| 20SB | | | ON | ON |
| 20SC | | ON | ON | |
| 20SD | ON | ON | | |

For example, when a joy stick constitutes input device 75 of operation unit 102, four directions of the joy stick may correspond to the rightward, backward, leftward, and frontward directions. In this case, control signal generator 74 of operation unit 102 may generate the control signal for controlling the drive voltage to be applied to each electrode structure 20 in accordance with correspondence shown in Table 1.

As indicated in the example described later, the force applied by EW is about 1 μN·m, and thus self-running is difficult by the force applied by EW. However, when capsule endoscope 101 is to be moved faster than peristaltic motion of a human body, the drive voltage is applied only to the upper part, or when capsule endoscope 101 is to be moved slowly against the peristaltic motion, the drive voltage is applied only to the lower part. In this manner, the movement speed with respect to the peristaltic motion of a human body can be changed.

As illustrated in FIG. 4 and Table 1, continuously changing the posture of capsule endoscope 101 in order of rightward, backward, leftward, and frontward allows capsule endoscope 101 to rotate counterclockwise when viewed from a traveling direction. For example, the drive voltage may be applied to the electrode structures in the following order:
1: 20NB, 20NC, 20SA, 20SD
2: 20NA, 20NB, 20SC, 20SD
3: 20NA, 20ND, 20SB, 20SC
2: 20NC, 20ND, 20SA, 20SB

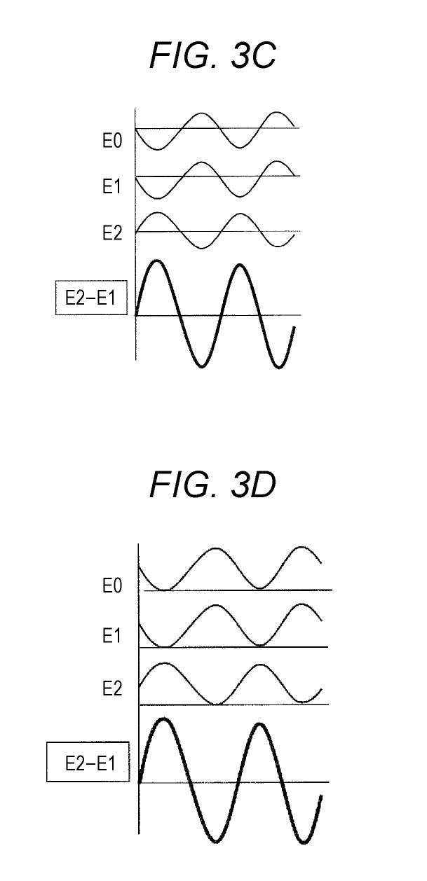

Next, reference electrode 22 will be described. When capsule endoscope 101 according to the present embodiment touches body fluid in the body, an EW drive circuit is formed. FIG. 5A illustrates how a human body becomes part of the EW drive circuit in a case where electrode structure 20 is distant from reference electrode 22. When reference electrode 22 is in contact with organ 32 of the subject via body fluid 30, organ 32, body fluid 30, and reference electrode 22 are maintained at common potential. However, when an electrostatic charge is discharged from electrode structure 20, or when a dielectric breakdown is produced in dielectric layer 26 and water repellent layer 27, an electric current, for example, of approximately several milliamperes may flow through organ 32 and body fluid 30. In addition, since organ 32 of a human body has resistance higher than resistance of body fluid 30, when the human body constitutes part of the drive circuit, a desired drive voltage may not be applied to electrode structure 20, and appropriate posture control may become difficult.

From these considerations, reference electrode 22 may be provided adjacent to electrode structure 20, as illustrated in FIG. 5B. In this case, electrode structure 20 and reference electrode 22 are connected by only body fluid 30, not via organ 32. Therefore, it is possible to avoid the electrostatic charge that is discharged from electrode structure 20 from flowing through organ 32.

When capsule endoscope 101 includes eight electrode structures 20 as illustrated in FIG. 2B, capsule endoscope 101 preferably includes two reference electrodes 22 arranged at both ends in the longitudinal direction of the external wall surface of capsule enclosure 10, respectively. Since each of eight electrode structures 20 is adjacent to one of reference electrodes 22 accordingly, electrode structure 20 and reference electrode 22 are connected by only body fluid 30, not via organ 32 in many cases.

As described above, capsule endoscope 101 according to the present embodiment is capable of controlling the posture by using electrowetting. Capsule endoscope 101 does not need to include a large power supply inside capsule endoscope 101 for posture control, because posture control by electrowetting is based on movement of the electrostatic charge and does not need large electric current. In addition, capsule endoscope 101 is excellent in low invasiveness, because it is not necessary to provide a mechanical component for generating a driving force, such as a blade or a screw, outside of capsule endoscope 101.

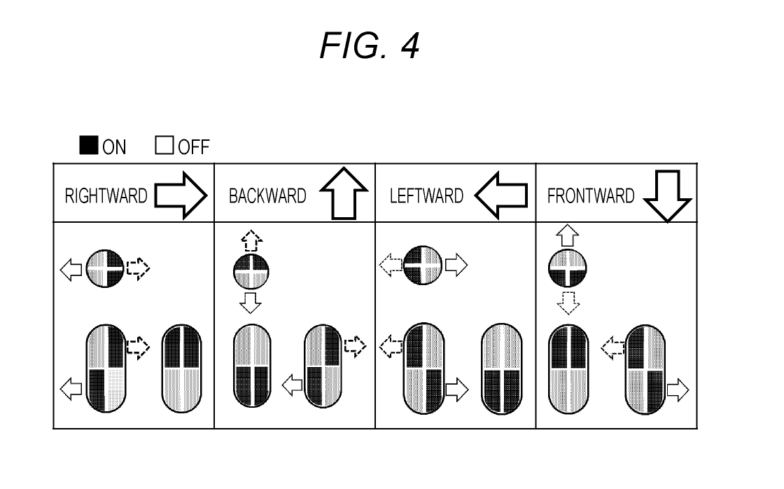

Although capsule endoscope 101 includes eight electrode structures in the present embodiment, capsule endoscope 101 including at least two electrode structures allows for posture control. For example, as illustrated in FIG. 2A, two electrode structures may be provided in two regions obtained by dividing capsule enclosure 10 with plane F1 perpendicular to the rotational axis, respectively. In addition, four electrode structures may be provided in a circumferential direction of the external wall surface in one of these two regions. Specifically, as illustrated in FIG. 6, first electrode structure 20N may be provided in one of the two regions obtained by dividing capsule enclosure 10 with plane F1 perpendicular to the rotational axis, and second to fifth electrode structures 205A to 20SD may be provided in the other region. Eight or more electrode structures may be provided.

In addition, all the electrode structures may have a common area, and may have different areas depending on where a center of gravity of capsule endoscope 101 is positioned. For example, the areas may differ between two electrode structures arranged in two regions obtained by dividing capsule endoscope 101 with the plane perpendicular to the rotational axis, respectively, such that a side on which the image pickup device is provided can be inclined more compared with an opposite side.

By suitably selecting a number, positions, areas, etc. of the electrode structures in this way, it becomes possible to change inclination and rotation of posture control, and a movement speed with respect to the peristaltic motion of a human body.

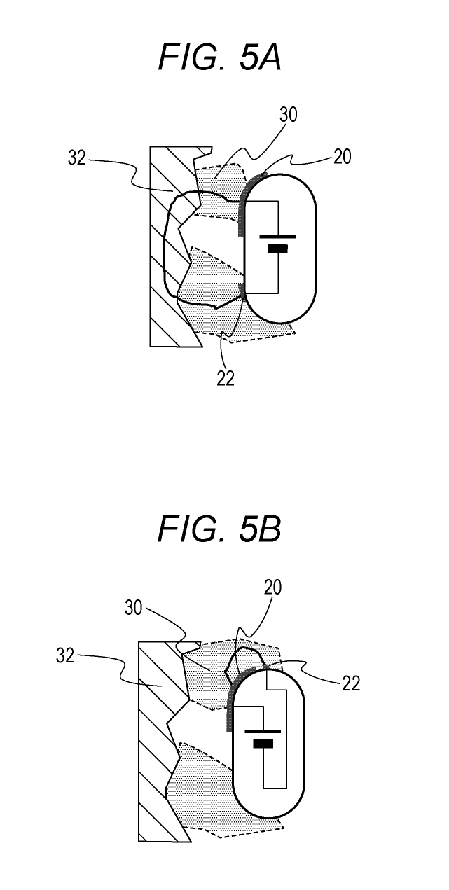

In addition, positions and a number of reference electrodes 22 are not limited to the above-mentioned embodiment. For example, capsule endoscope 101 illustrated in FIG. 7 has belt-shaped first, second, and third reference electrodes 22L1, 22L2, and 22L3. First, second, and third reference electrodes 22L1, 22L2, and 22L3 are positioned on first, second, and third planes F1, F2, and F3 illustrated in FIG. 2A, respectively. First, second, and third reference electrodes 22L1, 22L2, and 22L3 surround capsule enclosure 10 on first, second, and third planes F1, F2, and F3, respectively. According to this structure, each electrode structure is surrounded by the reference electrodes. Therefore, whatever posture capsule endoscope 101 has, electrode structure 20 and reference electrode 22 can be more securely connected only by body fluid 30, not via organ 32.

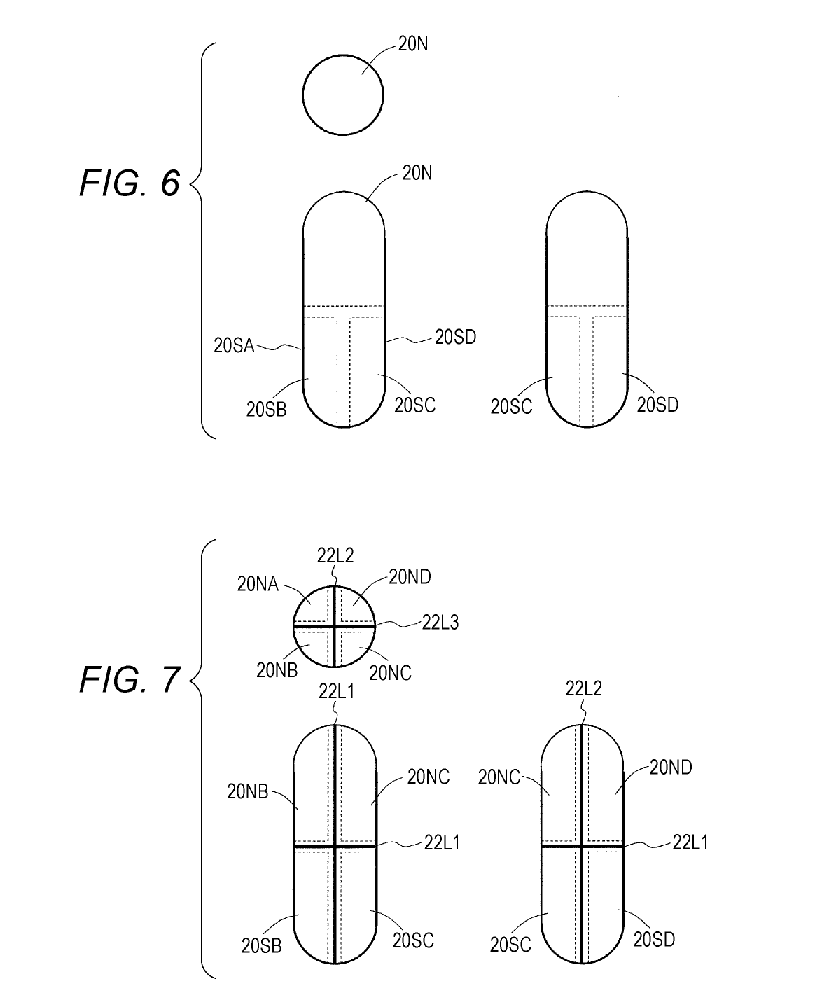

In addition, capsule endoscope 101 according to the present embodiment may include a mechanism for obtaining body fluid as a sample. As illustrated in FIG. 8A and FIG. 8B, capsule endoscope 101 includes sampling pipe 52 that has opening 52a. Electrode structure 54 having a structure similar to the structure of electrode structure 20 is provided inside sampling pipe 52. Specifically, electrode structure 54 includes electrode 55, water repellent layer 57, and dielectric layer 56 positioned between electrode 55 and water repellent layer 57. Electrode structure 54 is provided on an internal wall of sampling pipe 52 such that electrode 55 may be positioned on a side of the internal wall of sampling pipe 52.

Application of the drive voltage to electrode structure 54 allows hydrophilic properties inside sampling pipe 52 to be changed. For this reason, while an interior of the body of the subject is examined by using capsule endoscope 101, the drive voltage is applied to electrode structure 54 at a position of desired organ 32, so that the body fluid at the position can be collected by using a capillary phenomenon.

According to the present embodiment, the posture of capsule endoscope 101 is determined by an operator externally checking an image. However, in order to obtain the posture of capsule endoscope 101 automatically, capsule endoscope 101 may include a three-axis gyro sensor, for example. If capsule endoscope 101 includes the gyro sensor, controller 15 of capsule endoscope 101 may generate a control signal for generating the drive voltage to be applied to each electrode structure 20, in response to posture information obtained from the gyro sensor, such that the current posture coincides with a preset target posture. Controller 15 may then output the control signal to drive circuit 19.

### EXAMPLE

A result of estimating the force generated by electrowetting by experiment will be described below.

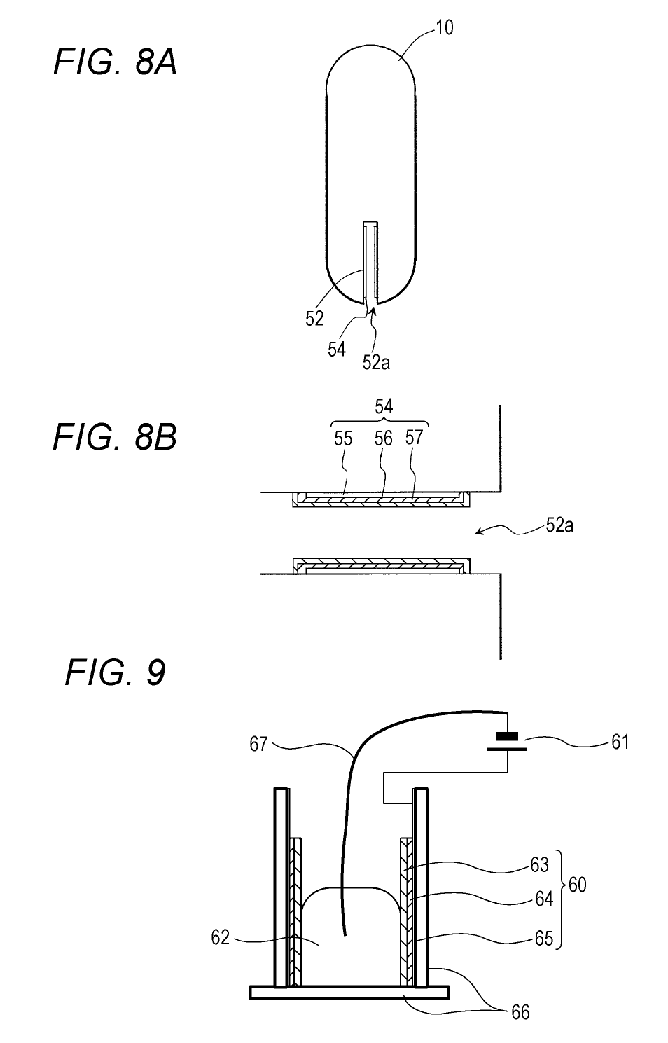

As an example, a cell illustrated in FIG. 9 was produced. An ITO film having a thickness of 100 nm was formed by a sputtering method across entire surface of glass substrate 66 having a size of 100 mm×100 mm as electrode 65. After part of glass substrate 66 is masked, an SiO₂ film having a thickness of 500 nm was formed by a sputtering method as dielectric layer 64. Two substrates each having a size of 20 mm×20 mm were cut out from this multilayer substrate.

Meanwhile, two untreated glass substrates each having a size of 10 mm×20 mm were prepared. These glass substrates and the above-mentioned multilayer substrates were adhered by using ultraviolet curable resin to produce a cylindrical glass cell.

Subsequently, water repellent layer 63 was formed in the cylindrical glass cell. CYTOP (produced by Asahi Glass Co., Ltd.) having a thickness of 1 μm and being formed by a dip coating method was used as water repellent layer 63. Subsequently, heat treatment was applied at 200° C. for one hour. Finally, glass substrate 66 having a size of 200 mm×300 mm was bonded as a bottom by using ultraviolet curable resin. Internal dimensions of the produced cell were 10 mm in width, 20 mm in height, and 20 mm in depth.

A voltage was applied by using DC power supply 61. Pure water 62 was poured as body fluid. Furthermore, a platinum wire was used as ground electrode 67.

A voltage was applied to electrode structure 60 from 0 V to 150 V in 10 V increments. In FIG. 9, a contact angle of pure water with respect to a right-hand glass substrate and liquid level variation were observed and measured.

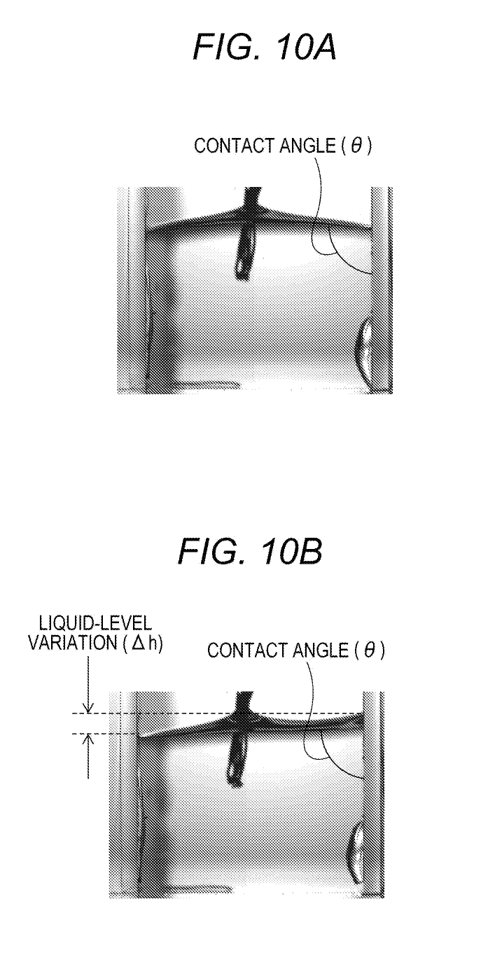

FIG. 10A and FIG. 10B illustrate states of pure water 62 in cases where the voltages of 0 V and 150 V were applied, respectively. Table 2 shows contact angles and liquid level variations.

TABLE 2

| | | |
|---|---|---|
| APPLIED VOLTAGE (V) | 0 | 150 |
| CONTACT ANGLE θ (°) | 107 | 67 |
| LIQUID LEVEL (mm) | 7.2 | 8.1 |
| LIQUID-LEVEL VARIATION (mm) | 0.9 | 0.9 |

γₛ=γ_{LS}+γ_{L}·cos(θ) [Equation 1]
γₛ surface tension of film
γ_{L} surface tension of liquid
γ_{LS} interfacial tension between solid and liquid

θ contact angle (Equation 1)

When the applied voltage is 0 V, the contact angle satisfies the Young equation (Equation 1). Since surface tension of pure water is 72.7 (mN/m) and surface tension of CYTOP is 19 (mN/m), interfacial tension between water and CYTOP can be estimated at 40 (mN/m).

Similarly, when the applied voltage is 150 V, assuming that the contact angle satisfies the Young equation (Equation 1), interfacial tension between water and CYTOP can be estimated at −9.4 (mN/m). Since interfacial tension cannot become negative, it is assumed that actually surface tension of pure water also changes. However, in order to estimate a force, the negative value was used as it was for convenience. It was estimated from this measurement result that a force capable of changing the interfacial tension between water and CYTOP from +40 (mN/m) to −9.4 (mN/m) was obtained by electrowetting.

At this time, an area wet with pure water has increased by only (depth of 20 mm)×(liquid level of 0.9 mm). It can be estimated that work increased by 0.89 (μN·m) by electrowetting before and after voltage application, from a value obtained by multiplying an amount of change in a wet area by an amount of change in interfacial tension. In addition, wetting of the liquid surface changes against gravity. When this is taken into consideration, actual force F by electrowetting can be estimated at 0.89 (μN·m) or more.

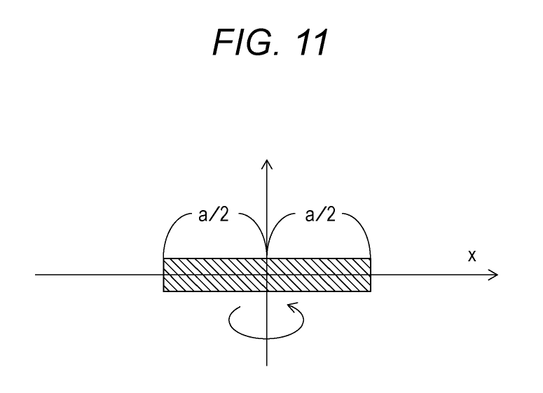

Meanwhile, posture control of the capsule endoscope can be estimated by the moment of inertia. FIG. 11 illustrates a rigid body that rotates around a rotational axis at a center of a bar, the rotational axis being perpendicular to the bar. The moment of inertia in this case can be calculated by (Equation 2). For example, assuming that the capsule endoscope is 30 mm in length and 30 g in weight, the moment of inertia of the bar is 1/12Ma², which is obtained by (Equation 2) and FIG. 11. Accordingly, the moment of inertia I of the capsule endoscope is 2.2×10⁻⁶ (kg·m²).

$$\begin{matrix}[ Equation 2 ] & \\ I = {\int }_{- a / 2}^{a / 2} {x}^{2} \cdot ρ \cdot d x = \frac{{a}^{3}}{12} ρ = \frac{1}{12} {Ma}^{2} \\ I moment of inertia \\ dx minute length of axis along bar \\ ρ surface density of bar , that is ρ \cdot a = M \\ M weight of bar \\ a length of bar & ( Equation 2 )\end{matrix}$$

F=I·ω [Equation 3]
F force (N·m)
I moment of inertia (kg·m²)

ω angular velocity (radian/s) (Equation 3)

Angular velocity that acts on the capsule endoscope was estimated by (Equation 3). Angular acceleration ω obtained from the moment of inertia I and the electrowetting force F was 0.397 (radian/s), that is, 22.7 (°/s).

Estimates from this result show that the capsule endoscope according to the present embodiment can be inclined several tens of degrees per second. Therefore, this indicates that the posture of the capsule endoscope can be controlled adequately by electrowetting. These values can be changed by adjusting the areas and positions of the electrode structures, and the drive voltage to be applied to the electrode structures, in accordance with the length and weight of the capsule endoscope.

The capsule endoscope disclosed in the present application is useful for obtaining information on a living body, such as a small intestine, that is difficult to access with an endoscope, such as a gastrocamera or a large intestine camera. The capsule endoscope disclosed in the present application makes it possible to achieve posture control of the capsule endoscope from outside of the body with low power consumption and low invasiveness. This facilitates obtaining of desired living body information, obtaining of the living body information inside a human body efficiently, and analysis of medical data.

### REFERENCE SINGS LIST

10 capsule enclosure

10a portion

14 image pickup device

15 controller

16 light source

17 wireless communicator

18 power supply

19 drive circuit

19a booster circuit

19b resistor

19c diode

19d relay

19e DC/AC converter

19f phase controller

20 electrode structure

22, 22L1, 22L2, 22L3 reference electrode

25 electrode

26 dielectric layer

27 water repellent layer

30 body fluid

32 organ

52 sampling pipe

52a opening

54 electrode structure

55 electrode

56 dielectric layer

57 water repellent layer

60 electrode structure

61 DC power supply

62 pure water

63 water repellent layer

64 dielectric layer

65 electrode

66 glass substrate

67 ground electrode
71 wireless communicator

72 image processor

73 display unit

74 control signal generator

75 input device

76 memory

77 wireless communicator

101 capsule endoscope

1. A capsule endoscope comprising:
a capsule enclosure comprising an external wall surface;
an image pickup device provided inside the capsule enclosure;
a light source provided inside the capsule enclosure;
a plurality of electrode structures each comprising an electrode, a water repellent layer, and a dielectric layer positioned between the electrode and the water repellent layer, the plurality of electrode structures being provided on the external wall surface of the capsule enclosure in such a manner that the electrode is positioned on an external wall surface side of the capsule enclosure;
a power supply provided inside the capsule enclosure;
at least one reference electrode provided on the external wall surface of the capsule enclosure and connected to reference potential of the power supply; and
a drive circuit configured to apply a drive voltage to the plurality of electrode structures based on the power supply.

2. The capsule endoscope according to claim 1, wherein the drive circuit changes hydrophilic properties on a surface of the water repellent layer of each of the electrode structures by controlling the drive voltage to be applied to the plurality of electrode structures.

3. The capsule endoscope according to claim 1, wherein
the external wall surface of the capsule enclosure comprises a longitudinal direction and a shape of a rotating body rotating around a rotational axis parallel to the longitudinal direction, and
the external wall surface comprises first and second regions divided by a plane perpendicular to the rotational axis.

4. The capsule endoscope according to claim 3, wherein the plurality of electrode structures comprise at least two electrode structures arranged in the first and second regions, respectively.

5. The capsule endoscope according to claim 3, wherein the plurality of electrode structures comprise:
a first electrode structure arranged in the first region; and
second, third, fourth, and fifth electrode structures arranged in a circumferential direction of the external wall surface in the second region.

6. The capsule endoscope according to claim 1, wherein
the external wall surface of the capsule enclosure comprises a longitudinal direction and a shape of a rotating body rotating around a rotational axis parallel to the longitudinal direction, and
the image pickup device is positioned on a one-end side in the longitudinal direction of the capsule enclosure.

7. The capsule endoscope according to claim 6, wherein
the external wall surface of the capsule enclosure comprises eight regions divided by a first plane perpendicular to the rotational axis, and by a second plane and a third plane comprising the rotational axis and being orthogonal to each other,
the plurality of electrode structures comprises:
first, second, third, and fourth electrode structures arranged in four regions on a side of the first plane where the image pickup device is positioned, respectively, in a clockwise order when the external wall surface of the capsule enclosure is viewed along the rotational axis from a side where the image pickup device is positioned; and
fifth, sixth, seventh, and eighth electrode structures arranged in four regions on an opposite side of the first plane from the image pickup device, the fifth, sixth, seventh, and eighth electrode structures being adjacent to the first, second, third, and fourth electrode structures, respectively.

8. The capsule endoscope according to claim 3, wherein
the at least one reference electrode comprises two reference electrodes, and
the two reference electrodes are arranged at both ends in the longitudinal direction of the external wall surface, respectively.

9. The capsule endoscope according to claim 7, wherein
the at least one reference electrode comprises belt-shaped first, second, and third reference electrodes, and
the first, second, and third reference electrodes are positioned on the first, second, and third planes, respectively.

10. The capsule endoscope according to claim 1, wherein the drive voltage is a direct-current voltage.

11. The capsule endoscope according to claim 10, wherein the drive circuit comprises:
a booster circuit configured to generate the drive voltage higher than a voltage of the power supply based on the power supply; and
a relay comprising a first end connected to a terminal to which the booster circuit outputs the drive voltage, and a second end connected to the electrode of each of the electrode structures.

12. The capsule endoscope according to claim 1, wherein the drive voltage is an alternating-current voltage.

13. The capsule endoscope according to claim 12, wherein the drive circuit comprises:
a DC/AC converter configured to generate the alternating-current voltage based on the power supply, and to apply the alternating-current voltage to each of the electrode structures; and
a phase controller configured to control a phase of the alternating-current voltage to be applied to each of the electrode structures.

14. The capsule endoscope according to claim 1, further comprising:
a sampling pipe provided inside the capsule enclosure, the sampling pipe comprising an opening on the external wall surface of the capsule enclosure; and
a different electrode structure comprising an electrode, a water repellent layer, and a dielectric layer positioned between the electrode and the water repellent layer, the different electrode structure being provided on an inner wall of the sampling pipe such that the electrode is positioned on the inner wall of the sampling pipe.

15. The capsule endoscope according to claim 1, further comprising a controller and a wireless communicator provided inside the capsule enclosure,
wherein the wireless communicator transmits image data obtained by the image pickup device to an external apparatus, and receives a control signal from the external apparatus, and
the controller drives the drive circuit in response to the control signal, and applies the drive voltage to the plurality of electrode structures selectively.

16. A capsule endoscope system comprising:
a capsule endoscope; and
an operation unit, wherein the capsule endoscope comprises:
a capsule enclosure comprising an external wall surface;
an image pickup device provided inside the capsule enclosure;
a light source provided inside the capsule enclosure;
a plurality of electrode structures each comprising an electrode, a water repellent layer, and a dielectric layer positioned between the electrode and the water repellent layer, the plurality of electrode structures being provided on the external wall surface of the capsule enclosure in such a manner that the electrode is positioned on an external wall surface side of the capsule enclosure;
a power supply provided inside the capsule enclosure;
at least one reference electrode provided on the external wall surface of the capsule enclosure and connected to reference potential of the power supply; and
a drive circuit configured to apply a drive voltage to the plurality of electrode structures based on the power supply;
the capsule endoscope further comprises a controller and a wireless communicator provided inside the capsule enclosure;
the wireless communicator transmits image data obtained by the image pickup device to an external apparatus, and receives a control signal from the external apparatus;
the controller drives the drive circuit in response to the control signal, and applies the drive voltage to the plurality of electrode structures selectively;
the capsule endoscope system comprises:
a different wireless communicator configured to receive the image data transmitted from the capsule endoscope and to transmit the control signal;
an image processor configured to apply image processing to the image data received by the different wireless communicator;
a display unit configured to display the image data that undergoes the image processing;
an input device configured to receive an input from an operator; and
a control signal generator configured to generate the control signal in response to the input to the input device.

17. A method for changing a posture of a capsule endoscope; the method comprising:
(a) administrating the capsule endoscope to a subject;
wherein the capsule endoscope comprises:
a capsule enclosure comprising an external wall surface;
an image pickup device provided inside the capsule enclosure;
a light source provided inside the capsule enclosure;
a first electrode comprising an electrode, a water repellent layer, and a dielectric layer positioned between the electrode and the water repellent layer, the first electrode structure being provided on the external wall surface of the capsule enclosure in such a manner that the electrode is positioned on an external wall surface side of the capsule enclosure;
a second electrode comprising an electrode, a water repellent layer, and a dielectric layer positioned between the electrode and the water repellent layer, the second electrode structure being provided on the external wall surface of the capsule enclosure in such a manner that the electrode is positioned on an external wall surface side of the capsule enclosure;
a power supply provided inside the capsule enclosure;
at least one reference electrode provided on the external wall surface of the capsule enclosure and connected to reference potential of the power supply; and
a drive circuit configured to apply a drive voltage to the plurality of electrode structures based on the power supply; and
(b) generating force along a direction from the second electrode toward the first electrode by applying, between the first electrode to the reference electrode, a voltage that is greater than a voltage applied between the first electrode and the reference electrode to change the posture of the capsule endoscope, while the first electrode, the reference electrode, and the second electrode are in contact with water on a surface of an internal periphery of a digestive organ of the subject.
